(19) United States
(12) Patent Application Publication
(10) Pub. No.: US 2019/0380301 A1
(43) Pub. Date: Dec. 19, 2019

(54) PET LOUNGING MAT

(71) Applicant: Craig Wallin, North Augusta, SC (US)

(72) Inventor: Craig Wallin, North Augusta, SC (US)

(21) Appl. No.: 16/012,489

(22) Filed: Jun. 19, 2018

(51) Int. Cl. A01K 1/035; A01K 1/015

(52) U.S. Cl. CPC A01K 1/0353; A61L 9/02; A01K 1/0157

(57) ABSTRACT

A mat that includes: a top surface; side panels; at least one zipper; an inside pocket; and at least one scented sheet adapted for placement within the inside pocket. The mat is preferably rectangular in shape. In one embodiment, the at least one zipper includes a first zipper at an end portion of the mat and a second zipper, which provides access to the inside pocket. The scented sheet releases a scent that is activated by body heat of a pet and provides a soothing environment for the pet while relaxing on a mat.

### BACKGROUND OF THE INVENTION

### Field of Invention

The present invention relates to a lounging mat designed for a pet such as a dog or cat.

### Description of Related Art

Various pet accessories are provided by the owners to enhance the pet's lifestyle. Typically, a pet such as a dog or a cat may have a small resting area to nap or sleep at night. Small comfortable pads and bedding have been developed to provide the pet with a particular location for lounging or sleeping. Nevertheless, most pets remain quite active and may not use such bedding areas as are desired by the pet owners. In general, pets are sensitive to their general and physical environment. In certain instances, if a pet travels out of town there may be some anxiety and uneasiness of the pet. This issue may present for pet owners who may be in a guest's home or traveling out of town in a relative's home. As a result, visiting pets and owners must be develop a system to address this problem. If a pet does become agitated or uncomfortable this could lead to negative consequences. Occasionally pets may excessively bark and destroy or scratch furniture if in a perceived stressful environment due to the new and unfamiliar environment.

The object of the present invention is to provide a mat that includes a inside pocket that responds to the pet's body heat. The body heat helps to trigger the relief of pheromone scents to soothe the animal during a stressful situation.

### SUMMARY OF THE INVENTION

The present invention relates to a mat that includes: a top surface; side panels; at least one zipper; an inside pocket; and at least one scented sheet adapted for placement within the inside pocket. The mat is preferably rectangular in shape. In one embodiment, the at least one zipper includes a first zipper at an end portion of the mat and a second zipper, which provides access to the inside pocket. The scented sheet releases a scent that is activated by body heat of a pet and provides a soothing environment for the pet while relaxing on a mat.

### BRIEF DESCRIPTION OF DRAWINGS

FIG. 1 depicts a pet mat in accordance with the present invention.

### DETAILED DESCRIPTION

The present invention relates to a mat adapted for use by a pet. The mat includes a flat smooth surface with an underlying inside pocket. Within this inside pocket, a scented sheet is inserted into the pocket, where the scented sheet releases a scent that is activated upon the pet's body heat.

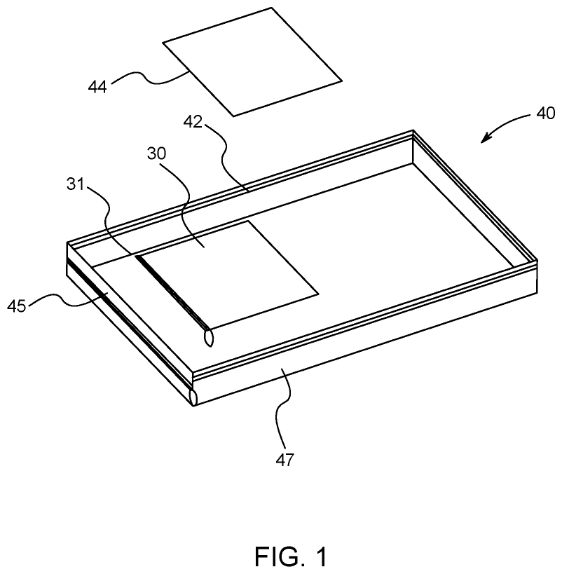

In reference to FIG. 1, a mat 40 in accordance with the present invention is shown. The mat 40 includes a top surface 42 and side panels 47. The mat 40 is essentially rectangular in shape and includes zippers, a first zipper 45 at an end portion of the mat and a second zipper 31, which provides access to an inside pocket 30. The inside pocket 30 is adapted to receive a scented sheet 44. The scented sheet 44 releases a scent that is activated upon the body heat of the pet. The sheet's 44 aroma provides a soothing environment for the pet while relaxing on a mat 40. A variety of scents may be infused in the sheet 44 and the sheet 44 provides aromatherapy to relax the pet resting on the mat 40. The top surface 42 of the mat 40 is preferably made of a fleece material and may have up to three layers of covering. Duck-type fabric or corduroy may used on the side panels 47 in FIG. 1.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A mat comprising:
a. a top surface;
b. side panels;
c. at least one zipper;
d. an inside pocket; and
e. at least one scented sheet adapted for placement within the inside pocket.

2. The mat according to claim 1, where the mat is rectangular in shape.

3. The mat according to claim 1, where the at least one zipper includes a first zipper at an end portion of the mat and a second zipper, which provides access to the inside pocket.

4. The mat according to claim 1, where the scented sheet releases a scent that is activated by body heat of a pet.

5. The mat according to claim 1, the sheet aroma provides a soothing environment for the pet while relaxing on a mat.
